(19) United States
(12) Patent Application Publication
(10) Pub. No.: US 2012/0171312 A1
(43) Pub. Date: Jul. 5, 2012

(54) THERAPY OF NON ALCOHOLIC FATTY LIVER DISEASE UTILIZING INULA VISCOSA EXTRACT

(75) Inventors: Maria Grozovski, Karmiel (IL); Michal Maoz, Karmiel (IL); Nimer Assy, Fassouta (IL)

(73) Assignee: OFEK ESHKOLOT RESEARCH AND DEVELOPMENT LTD., Karmiel (IL)

(21) Appl. No.: 13/383,583

(22) PCT Filed: Jun. 9, 2010
(86) PCT No.: PCT/IL10/00454

§ 371 Date: Mar. 15, 2012

(30) Foreign Application Priority Data
Jul. 14, 2009 (IL) 199847

(51) Int. Cl. A61K 36/28; A61P 1/16

(52) U.S. Cl. 424/764

(57) ABSTRACT

The invention describes a substance and method for treating non alcoholic steatohepatitis (NASH) and other non alcoholic fatty liver disease (NAFLD) in a mammal in need thereof, the method comprises of administering to the mammal in need thereof a therapeutically effective amount of an extract derived from the Inula viscosa plant for reducing of hepatic concentrations of cholesterol, protein C, malonic dialdehyde and triglycerides and for increasing the activity of hepatic antioxidants.

### FIELD AND BACKGROUND OF THE INVENTION

This invention relates to the area of exploiting therapeutically properties of compositions extracted from naturally occurring substances and more particularly, to a substance and method for decreasing the hepatic triglycerides and increasing antioxidants levels.

Normally, less than 5% of the liver volume is fat, but in patients with non alcoholic steatohepatitis (NASH) or non alcoholic fatty liver diseases (NAFLD), up to 50%-80% of liver weight may be made up of fat, mostly in the form of triglycerides, see e.g.: Sanyal, A. J. in “AGA technical review on non-alcoholic fatty liver disease”, Gastroenterology 2002; 123, 1705-1725.

The clinical implications of fat accumulation in the liver are derived mostly from its common occurrence in the general population (10-24%) and its potential to progress to fibrosis (40%), cirrhosis (30%) and hepatocellular carcinoma, see e.g.: Chitturi et al. in “NASH and insulin resistance, insulin hypersecretion and specific association with the insulin resistance syndrome”, Hepatology 2002; 35, 373-379.

NAFLD is the most common cause of cryptogenic cirrhosis and is an increasingly common indication for liver transplantation, see e.g.: Musso et al. in “Dietary habits and their relations to insulin resistance and postprandial lipemia in non-alcoholic steatohepatitis”. Hepatology, 2003; 37: 909-916.

NAFLD is a component of the metabolic insulin resistance syndrome, with a clinical spectrum ranging from simple fatty liver to steatohepatitis, bridging fibrosis and cirrhosis, see e.g.: Angulo et al., in “Independent predictors of liver fibrosis in patients with non-alcoholic steatohepatitis”, Hepatology 1999; 30, 1356-1362.

Obesity and diabetes type 2 are considered the most powerful predisposing risk factors for the development of more severe manifestation of NAFLD which like NASH, is bridging fibrosis and cirrhosis, see e.g.: Day et al. in “A tale of two hits”, Gastroenterology 1998; 114, 842-845 and Loguercio et al., in “Non-alcoholic liver disease in an area of Southern Italy: Main clinical, histological and pathophysiological aspects”, J. Hepatol 2000; 35, 568-574.

The pathogenesis of NASH is multifactorial; the primary event of NASH is the accumulation of triglyceride in hepatocytes which seems to be determined by insulin resistance.

These fat stem mainly from increased splanchnic lipolysis of visceral fat (70%), and from continuous delivery of free fatty acids to the liver after ingestion of fatty foods (30%), both of which increase also hepatic insulin resistance, see e.g.: MacDonald, et al., in “Lipid peroxidation in hepatic steatosis in humans is associated with hepatic fibrosis and occurs predominantly in acinar zone 3”, J. Gastroenterol Hepatol 2001; 16, 599-606.

The secondary event is hepatocellular injuries which include factors such as oxidative stress which results in significant lipid peroxidation which is accompanied by a significant increase in the concentration of malonic dialdehyde (MDA) in the liver, see e.g.: Ackerman et al., in “Effects of amlodipine, captopril and bezafibrate on oxidative milieu in rats with fatty liver”, Journal Digestive Diseases and Sciences, 53 (3); 777-784, 2008, and proinflammatory cytokines, mitochondrial dysfunction, iron overload, bacterial overgrowth and genetic predisposition, see e.g.: Seki et al., in “In situ detection of lipid peroxidation and oxidative DNA damage in non-alcoholic fatty liver disease”, J. Hepatol 2002; 37, 56-62.

In light of these findings, it seems possible that the reduction of fat absorption, reduction of oxidative stress, reduction of inflammation as well as the use of insulin sensitizing agents may be successful treatment for NAFLD.

Previous attempts to treat NAFLD including the use of ursodeoxycholic acid see e.g.: Malaguarnera et al., in “Heme-oxygenase-1 levels and oxidative stress-related parameters in non-alcoholic fatty liver disease patients”, J. Hepatol 2005; 42, 585-591 and Bahcecioglu et al., in “Levels of serum vitamin A, alpha-tocopherol and malondialdehyde in patients with non-alcoholic steatohepatitis: Relationship with histopathologic severity”, Int. J. Clin. Pract., 2005; 59, 318-323 and Hussein et al., in “Monounsaturated fat decrease hepatic lipid content in non alcoholic fatty liver disease in rats”, World Journal of Gastroenterology, 2007; 13(3), 361-368, were disappointing.

Recently there has been increased interest in research of the role of antioxidant materials in the prevention and treatment of patients with NAFLD.

Altered antioxidants and lipid peroxidation in NAFLD patients has been shown in previous studies. Administration of certain antioxidants such as glutathione (GSH), S-adenosyl methionine and vitamin E ameliorate the severity of ethanol-induced liver damage in rats, see e.g.: Mato, et al., in “S-denosylmethionine a control switch that regulates liver function”, FASEB 2002; 16, 15-26.

A part from pharmacological intervention, another potential measure could be to increase the dietary intake of flavonoids, compounds that are exceptionally efficient antioxidants and radical scavengers such as Fenugreek seed polyphenolic extract which significantly reduces the levels of lipid peroxidation products and increases the activities of antioxidant enzymes in ethanol-fed rats, see e.g.: Kaviarasan et al., in “Fenugreek seed polyphenols protect liver from alcohol toxicity: a role on hepatic detoxification system and apoptosis”, Pharmazie, 62 (4), 299-304, 2007 and Spanish needles herb extract see e.g.: Application CN2005010097344 to Chen and JP208195672 to Takayama et al.

In spite these efforts, to date no effective and consistent therapy for fatty liver disease has been identified. Thus there exist a need for a composition which will enable curing or alleviation the syndromes of NAFLD and NASH.

The present invention fulfils this need and carries related advantages.

### SUMMARY OF THE INVENTION

The present invention comprises a method for reducing the concentration level of hepatic lipids which exist in mammals with non alcoholic steatohepatitis (NASH) or non alcoholic fatty liver diseases (NAFLD), by administering to the mammals in need an extract derived from the Inula viscosa plant.

In accordance with the present invention there is provided a method for treating non alcoholic steatohepatitis (NASH) or non alcoholic fatty liver diseases (NAFLD) in a mammal in need thereof; the method comprises of administering to the mammal in need thereof a therapeutically effective amount of an extract derived from Inula viscosa plant for achieving one or more of the following changes in hepatic lipid levels of said mammal: (i) reduction of concentrations of cholesterol and protein C, (ii) reduction the level of MDA and (iii) reduction of concentration of triglyceride.

It is a further aim of the present invention to provide a method for increasing the hepatic activity of antioxidants such as alpha tocopherol and paroxonase.

In accordance with the present invention there is provided a composition effective in treating non alcoholic steatohepatitis (NASH) and other non alcoholic fatty liver disease (NAFLD) in a mammal in need thereof, comprising an extract derived from the leaves of the Inula viscosa plant wherein administering the extract results in achieving one or more of the following changes in hepatic lipid levels of said mammal: (i) reduction of concentrations of cholesterol and protein C, (ii) reduction of level of MDA and (iii) reduction of concentration of triglyceride.

It is a further object of the present invention to provide a composition for increasing the hepatic activity of antioxidants such as alpha tocopherol and paraoxonase.

Other advantages and benefits of the invention will become apparent upon reading its forthcoming description.

### DETAILED DESCRIPTION OF PREFERRED EMBODIMENTS

The present embodiments herein are not intended to be exhaustive and to limit in any way the scope of the invention; rather they are used as examples for the clarification of the invention and for enabling of other skilled in the art to utilize its teaching.

The present invention is a method for reducing hepatic cholesterol concentrations, hepatic triglyceride concentration, hepatic protein C concentrations and hepatic levels of MDA which exist in mammals with non alcoholic steatohepatitis (NASH) or non alcoholic fatty liver diseases (NAFLD), by administering to the mammals in need an extract of the Inula viscosa plant.

Inula viscosa (helenium), which is a member of the Compositae family, is a plant that grows to about 1 to 1.5 meter in height. The leaves and stems of the plant are coated with a sticky resin. Its flowers which blossom mainly during August to November are widely rounded and are yellowish in color. Roots are deep brown in color, whitish on the inside and have a characteristic smell. The plant is also known in Arabic as Rasen. This plant grows in the Mediterranean basin.

Extract of Inula viscosa is prepared according to the method developed by Michal Maoz: see e.g.: Maoz M. et al., in “Isolation and identification of a new antifungal Sesquiterpene Lactone from Inula viscose”, Planta Medica 65, 1999, which is incorporated here by reference for all purposes as if fully set forth herein.

Preferably, leaves of Inula viscosa are collected at the end of summer in the Galilee, Israel and dried immediately after collection at an oven at 60° C. for 14 hours and then the dried leaves are grinded to powder.

The powdered leaves are extracted with a borate buffer pH 9.0 (0.1M boric acid with the addition of NaOH). The concentration of the dried powdered leaves in the buffer is about 10% in weight (w %). The extraction was carried out in an autoclave at 121° C. for 15 minutes to get an aqueous extract.

After cooling, the aqueous extract was filtered through few layers of gauze. This filtrate was used as the source of the Inula viscosa extract in the experiments which are described below and will be referred to hereinafter as the Inula viscosa extract.

Inula viscosa extract is known for its biological activity. It is active against microorganism, especially fungi, see e.g.: Maoz, M. and Neeman I., in “Effect of Inula viscosa extract on chitin synthesis in dermatophytes and Candida albicans”, Journal of Ethnopharmacology 71; 479-482, 2000 and U.S. Pat. No. 4,254,112 to Debat et al.

Other kinds of extract of Inula viscosa plant possess antiviral properties see e.g.: U.S. Pat. No. 6,841,174 to Shalaby et al. and are effective in reduction blood glucose see e.g.: Zeggwaghaet al., “Study of hypoglycaemic and have cytotoxic effect on several cancer cell cultures, hypolipidemic effects of Inula viscosa L. aqueous extract in normal and diabetic rats”, J. of Ethnopharmacology, Vol. 108, issue 2, 223-227, 2006, and has cytotoxic effect on several cancer cell cultures.

The activity of Inula viscosa extract is attributed to the compound-tayunin which is a sesquiterpen lacton, see e.g.: “Tayunin—A new compound from Inula viscosa leaves and its antifungal activity against dermatophytes and the yeast Candida albicans”, A dissertation of the Faculty of Food Engineering and Biotechnology, Technion-IIT, Haifa, Israel 1997, and Berdicevski et al., in “Antimycotic Activity of Tayunin-Inula Viscosa Extract-SEM Observations”, 41st Interscience Conference on Antimicrobial Agents and Chemotherapy, Chicago Ill., 2001.

Inventors of the present invention disclosed an effective reduction of hepatic cholesterol concentration, hepatic triglyceride concentration, hepatic protein C concentration and hepatic levels of MDA together with an increase of hepatic antioxidant activity in mammals suffering non alcoholic steatohepatitis (NASH) and other non alcoholic fatty liver disease (NAFLD) which is achieved by administrating to the sick mammals the Inula viscosa extract.

This disclosure is based on carefully designed laboratory tests in a study which was conducted using rats and which is described below.

Procedures Used in Carrying Out the Study

Twenty four male Sprague-Dawley rats (Harlan Laboratories Ltd., Jerusalem, Israel) weighing 170±20 grams were studied. Rats were housed in regular cages situated in an animal room at 22° C., with a 14/10-hour light/dark cycle. Rats were maintained on standard rat chow diet (SRCD) (pellets #19520; Koffolk, Tel Aviv, Israel) and were given tap water to drink ad libitum.

All animal studies were conducted according to the regulations for the use and care of experimental animals.

At the beginning of our study rats were randomly divided in two groups. One group (12 rats) served as the control group and was maintained on standard chow diet and its members were given tap water to drink ad libitum for 12 weeks, whereas the other group of 12 rats which serves as the target group was given fructose enriched diet (FED) only (TD 89247; Harlan Teklad, Madison, Wis., USA) for 12 weeks.

The FED contained (as supplied by Harlan Teklad) 20.7% (per weight basis) protein (as casein), 5% fat (as lard), 60% carbohydrates (as fructose), 8% cellulose, 5% mineral mix (#170760; R-H) and 1% vitamin mix (#40060; Teklad). The SRCD (Koffolk) contains 21.9% protein, 4.5% fat, 41% starch, 5% sugar and 3.7% crude fiber. Both diets were in pellet form.

Hepatic lipid extraction: Total hepatic lipids were extracted from freeze-dried liver samples by chloroform: methanol (2:1) according to Folch et al. (Folch et al. in “A simple method for isolation and purification of total lipids from animal tissues.” J. Biol. Chem.; 226, 497-509 1957.

Hepatic enzymes extraction: Hepatic antioxidant enzymes were measured in the rat liver cytosolic fraction. Approximately 0.5 g of liver were homogenized in 5 ml of ice-cold 50 mmol phosphate buffer (pH 7.4). The liver homogenate was centrifuged for 10 minutes at 5000 rpm at 4° C. and the pellet was discarded. The supernatant was centrifuged for a further 30 minutes at 8000 rpm at 4° C.

Effect of Fructose Diet on Rats

Fatty liver induced by the fructose enriched diet (FED) had +10% increases in the hepatic cholesterol concentrations as compared to control liver. Hepatic triglyceride, hepatic protein C concentration and hepatic levels of MDA were significantly higher (+240%, +58.7% and +109% respectively). Fatty liver rats had significantly lower concentrations of alpha-tocopherol and paraoxonase activity when compared with the liver of the control group (−30% and −47%, respectively).

Both the control group and the target group (the FED rats) were randomized and divided into two groups having two treatments with six repetitions as shown in Table 1.

TABLE 1
Treatments groups

| Group 1 | Group 2 |
|---|---|
| No | Inula viscosa |
| treatment | extract |

The treatment began 12 weeks after the initiation of the FED and lasted for 4 weeks.

Group 1 remained without any treatment while group 2 was given 5.6 mg per day of the Inula viscosa extract.

The Inula viscosa extract was administered orally as a medicament via the drinking water of the rats, yet it can also wet and soak the rat solid food and thus be administered as a food supplement.

1. The Effect of the Treatments on Control Rats

The findings are shown in tables 2-4 below and summarized as follows:

1.1 Inula viscosa extract reduced hepatic cholesterol and protein C by −8% and −3% respectively and increased hepatic level of MDA (+5%) as compared to untreated group. Inula viscosa extract had increased concentrations of hepatic triglyceride, alpha tocopherol and paraoxonase (PON) (+6.5%, +54%, +9%, respectively), compared to the untreated group.

TABLE 2
Average values of hepatic cholesterol and hepatic triglycerides
in control rats as a result of the treatments

| | | Cholesterol | Variation | Triglycerides | Variation |
|---|---|---|---|---|---|
| Groups | | (mg/gr liver) | (%) | (mM/gr liver) | (%) |
| 1 | untreated | 1.32 ± 0.20 | | 7.80 ± 0.43 | |
| 2 | Inula viscosa | 1.21 ± 0.14 | −8.0 | 8.31 ± 0.79 | +6.5 |
| | extract | | | | |

TABLE 3
Average values of hepatic Protein C and hepatic malon dialdehyde
in control rats as a result of the treatments

| | | Protein C, | | malon | |
|---|---|---|---|---|---|
| | | Units/mg | Variation | dialdehyde, | Variation |
| Groups | | protein | (%) | (μM/gr liver) | (%) |
| 1 | untreated | 2.08 ± 0.30 | | 17.69 ± 1.63 | |
| 2 | Inula viscosa | 2.02 ± 0.19 | −3.0 | 18.50 ± 2.40 | +4.5 |
| | extract | | | | |

TABLE 4
Average values of hepatic alpha tocopherol and Paraoxonase
activity in control rats as a result of the treatments

| | | | | Paraoxonase | |
|---|---|---|---|---|---|
| | | alpha | | activity | |
| | | tocopherol, | Variation | μM/(min * mg | Variation |
| Groups | | (mg/gr liver) | (%) | protein) | (%) |
| 1 | untreated | 0.24 ± 0.05 | | 381.13 ± 89.11 | |
| 2 | Inula viscosa | 0.37 ± 0.03 | +54.0 | 416.66 ± 36.10 | +9.0 |
| | extract | | | | |

2. The Effect of the Treatments on FED Rats

Results are shown in tables 5-7 below and are summarized as follows:

2.1 Inula viscosa extract reduced hepatic triglyceride, hepatic cholesterol and protein C by −34%, −31% and −43% respectively and the hepatic levels of MDA were significantly lower (−49%) as compared to untreated group. Inula viscosa extract had significantly increased concentrations of alpha-tocopherol and paraoxonase activity (+94% and +97%, respectively).

TABLE 5
Average values of hepatic cholesterol and hepatic triglycerides
in FED rats as a result of the treatments

| | | | | Triglycerides | |
|---|---|---|---|---|---|
| | | Cholesterol | Variation | (mM/gr | Variation |
| Groups | | (mg/gr liver) | (%) | liver) | (%) |
| 1 | untreated | 1.45 ± 0.08 | | 26.49 ± 3.18 | |
| 2 | Inula viscosa | 1.00 ± 0.03 | −31.0 | 17.61 ± 1.08 | −34.0 |
| | extract | | | | |

TABLE 6
Average values of hepatic Protein C and hepatic malon dialdehyde
in FED rats as a result of the treatments

| | | Protein C, | | malon | |
|---|---|---|---|---|---|
| | | (Units/mg | Variation | dialdehyde, | Variation |
| Groups | | protein) | (%) | (μM/gr liver) | (%) |
| 1 | untreated | 3.30 ± 0.30 | | 37.0 ± 3.0 | |
| 2 | Inula viscosa | 1.88 ± 0.28 | −43.0 | 18.80 ± 1.40 | −49.0 |
| | extract | | | | |

TABLE 7
Average values of hepatic alpha tocopherol and Paraoxonase
activity in FED rats as a result of the treatments

| | | | | Paraoxonase | |
|---|---|---|---|---|---|
| | | alpha | | activity | |
| | | tocopherol | Variation | (μM/min * mg | Variation |
| Groups | | (mg/gr liver) | (%) | protein) | (%) |
| 1 | untreated | 0.17 ± 0.01 | | 203.51 ± 54.12 | |
| 2 | Inula viscosa | 0.33 ± 0.02 | +94.0 | 401.94 ± 95.41 | +97.5 |
| | extract | | | | |

Discussion of the Results of the Study

The observed redox imbalance in NAFLD as a consequence of decreased levels of antioxidants, along with an increased MDA levels in circulation may be an important factor in the development of NASH.

Our results showed that Inula viscosa extract had no effect on control healthy rats except increasing the antioxidant level.

In sick rats however, it succeeded to decrease cholesterol and MDA concentrations and to increase antioxidant concentration to the level of the control group. It also decreased the triglyceride (TG) level in sick rats.

Protein C is a sign for inflammation. It can be lowered too in sick rats by the extract. In healthy rats there is no impact of the extract with regard to protein C level.

The accumulation triglycerides and cholesterol in liver cells directly contributes to an atherogenic serum lipid profile (i.e. an increase of triglycerides and cholesterol in blood), see e.g. Ijaz-ul-Haque et al. in: “Frequency of non alcoholic fatty liver disease and its biochemical derangements in type-2 diabetic patients”, Pak J Med Sci (Part-I), Vol. 25 (5), 817-820 (2009), Kantartzis K, et al. in: “Fatty liver is independently associated with alterations in circulating HDL₂ and HDL₃ subfractions”, Diabetes Care 31(2), 366-368 (2008) and Ackerman Z. et al. in: “Fructose-induced fatty liver disease: Hepatic effects of blood pressure and plasma triglycerides reduction”, Hypertension, 45(5): 1012-1018, (2005).

Thus, reducing the concentration of liver cholesterol and triglyceride levels in rats with fatty liver by administering the extract derived from the leaves of the Inula viscose plant may also reduce the concentration of cholesterol and triglycerides in the blood of these rats.

While the invention has been described with respect to a limited number of embodiments, it will be appreciated that many variations, modifications and other applications of the invention may be made without departing from the spirit and scope of the invention.

What is claimed is:

1. A method for treating non alcoholic steatohepatitis (NASH) and other non alcoholic fatty liver disease (NAFLD) in a mammal in need thereof, the method comprises of administering to the mammal in need thereof a therapeutically effective amount of an extract derived from an Inula viscosa plant for achieving a change in state of the mammal liver selected from the group consisting of reduction of hepatic concentration of cholesterol, reduction of hepatic concentration of protein C, reduction of hepatic concentration of triglyceride and a combination thereof.

2. The method as claimed in claim 1, further achieving one or more of the following changes:
(i) reduction of hepatic level of MDA,
(ii) increasing the hepatic level of alpha-tocopherol and
(iii) increasing the hepatic activity of paraoxonase.

3. The method as claimed in claim 1, wherein said mammal is a rat.

4. The method as claimed in claim 1, wherein said extract is obtained from leaves of the Inula viscosa plant.

5. The method as claimed in claim 1, wherein said administering of said extract includes an oral administering of said extract.

6. The method as claimed in claim 5, wherein said extract is formulated as a medicament.

7. The method as claimed in claim 5, wherein said extract is formulated as a food additive.

8. The method as claimed in claim 5, wherein a daily therapeutically effective amount of said extract is about 35 mg per kg of body weight of said mammal in need.

9. A composition effective in treating non alcoholic steatohepatitis (NASH) and other non alcoholic fatty liver disease (NAFLD) in a mammal in need thereof, comprising an extract derived from the leaves of the Inula viscosa plant, the extract is effective in achieving a change in hepatic lipid levels of said mammal selected from the group consisting of reduction of hepatic concentration of cholesterol, reduction of hepatic concentration of protein C, reduction of hepatic concentration of triglyceride and a combination thereof.

10. The composition as claimed in claim 9, effective in further achieving one or more of the following changes:
(i) reduction of hepatic level of MDA,
(ii) increasing the hepatic level of tocopherol and
(iii) increasing the hepatic activity of paraoxonase.

11. The composition as claimed in claim 9, wherein said mammal is a rat.

12. The composition as claimed in claim 9, wherein said leaves are extracted in water at a temperature which is about 120° C.

13. The composition as claimed in claim 9, wherein administering of said extract includes an oral administering.

14. The composition as claimed in claim 13, wherein said extract is formulated as a medicament.

15. The composition as claimed in claim 13, wherein said extract is formulated as a food additive.

16. The composition as claimed in claim 9, wherein a daily therapeutically effective amount of said extract is about 35 mg per kg of body weight of said mammal in need.

17. A composition effective in treating an atherogenic serum lipid profile in a mammal in need thereof, comprising an extract derived from the leaves of the Inula viscosa plant, the extract is effective in achieving a change in blood lipid levels of said mammal selected from the group consisting of reduction of blood concentration of cholesterol, reduction of blood concentration of triglyceride and a combination thereof.

18. The composition as claimed in claim 17, wherein said mammal is a rat.

19. The composition as claimed in claim 17, wherein said leaves are extracted in water at a temperature which is about 120° C.

20. The composition as claimed in claim 17, wherein administering of said extract includes an oral administering.

21. The composition as claimed in claim 20, wherein said extract is formulated as a medicament.

22. The composition as claimed in claim 20, wherein said extract is formulated as a food additive.

23. The composition as claimed in claim 17, wherein a daily therapeutically effective amount of said extract is about 35 mg per kg of body weight of said mammal in need.
